(19) United States
(12) Patent Application Publication
(10) Pub. No.: US 2008/0281256 A1
(43) Pub. Date: Nov. 13, 2008

(54) Centrifuge Functioning as Liposuction and Lipoinjection and Method Using the Same

(76) Inventor: HeeYoung Lee, Cholrabukdo (KR)

Correspondence Address: SUMMA, ALLAN & ADDITON, P.A., 11610 NORTH COMMUNITY HOUSE ROAD, SUITE 200, CHARLOTTE, NC, 28277, US

(21) Appl. No.: 10/596,278

(22) PCT Filed: Apr. 18, 2005
(86) PCT No.: PCT/KR2005/001108

§ 371 Date: Jun. 7, 2006

(30) Foreign Application Priority Data
Apr. 19, 2004 (KR) 20-2004-0010685

(51) Int. Cl. A61M 1/00; B04B 15/08

(52) U.S. Cl. 604/28; 494/61

(57) ABSTRACT

Disclosed herein is a centrifuge having liposuction and lipoinjection functions. The centrifuge comprises an air pump for vacuum and compression, valves, and a controller, thereby sequentially performing liposuction, centrifugation, and lipoinjection processes for autologous fat grafting using a single instrument. More specifically, the centrifuge comprises a pump device consisting of the air pump mounted in a case of the centrifuge for vacuum and compression and the valves connected to the air pump and adapted to be automatically opened or closed to selectively perform vacuum or compression, the controller to control operation of the pump device, and a connector formed at a side of the case to connect the pump device with an external unit for performing vacuum and compression.

### TECHNICAL FIELD

The present invention relates to centrifuges having liposuction and lipoinjection functions, and more particularly to centrifuges having liposuction and lipoinjection functions, which comprise an air pump for vacuum and compression, valves and a controller, thereby sequentially performing liposuction, centrifugation, and lipoinjection processes for autologous fat grafting using a single instrument.

### BACKGROUND ART

In general, cosmetic surgery, such as breast enlargement surgery, and face-lift surgery performed on nasal and nasolabial regions, cheek, etc., involved a prosthesis insertion and autologous fat grafting. Prosthesis insertion allows easy enlargement or reduction of the size of a desired region of the body, but has a problem that it is clinically dangerous and may cause the user discomfort due to the unnatural sensation of having a foreign object inserted into the body. Autologous fat grafting comprises suctioning extra fat from the abdomen, legs, etc. of a patient and injecting the suctioned fat into the region of the patient's body that is to be enlarged, e.g. the breasts, hips and face. Autologous fat grafting enables not only the removal of unwanted fat, but also the enlargement of relatively gaunt body regions, thereby achieving a balanced figure and eliminating the problems associated with the insertion of a foreign object into the body. However, a problem of autologous fat grafting is that it must be performed two or three times to obtain a desired level of enlargement because the autologous fat shows an extremely low survival rate of 20-50%, resulting in massive time and cost. Therefore, there have been many attempts to increase the survival rate of autologous fat, and as a result of clinical experimentations, it has been found that the survival rate of autologous fat can be increased by obtaining pure fat and injecting it into the desired body regions to be enlarged. As is known, pure fat can be obtained by means of centrifuges that centrifugally separate fat from blood, bodily fluids, and other impurities suctioned along with the fat.

Conventional autologous fat grafting consists of liposuction, centrifugation, and lipoinjection processes. The liposuction process is normally performed by means of a suction device that is adapted to suction fat into a vacuum bottle. A centrifuge is used in the centrifugation process to obtain pure fat. Generally, the separated pure fat is manually injected into the desired body regions to be enlarged by means of a syringe.

As will be easily understood from the above description, conventional autologous fat grafting has problems in that it utilizes a plurality of devices including the centrifuge and the suction device, occupying too much space of an operation theater, and that it is inconvenient in use because an operator must frequently move about in order to operate the plurality of devices. Further, manual injection of the fat into specific body regions, for example, muscularis, that are reluctant to the insertion of a syringe, is very troublesome, and has the risk of causing unevenness of the fat injected body regions because it is substantially impossible to continuously inject the fat by hand at a constant pressure.

### Disclosure of Invention

### Technical Problem

Therefore, the present invention has been made in view of the above problems, and it is an object of the present invention to provide a centrifuge which comprises an air pump for vacuum and compression, valves, and a controller to allow an external unit to selectively perform vacuum or compression using a pneumatic pressure in addition to performing basic centrifugation, thereby ensuring convenient liposuction, centrifugation and lipoinjection for rapid and convenient autologous fat grafting as well as easy pressure adjustment, and consequently eliminating the risk of unevenness in the resultant fat suctioned or injected body regions.

Although some existing centrifuges have been conventionally provided with an air pump to produce a vacuum state within a chamber thereof to thereby reduce an air frictional force, the present invention has an outstanding feature that an air pump is connected to an external unit to impart vacuum and compression thereto to, thereby integrate the external unit with the centrifuge, rather than be used to produce such a vacuum state within a centrifuge chamber.

### Technical Solution

In accordance with an aspect of the present invention, the above and other objects can be accomplished by the provision of a centrifuge comprising: a centrifugal device having buckets, a rotor, and a chamber; a drive unit to drive the rotor; a controller to control the drive unit; a power supply to supply power to the drive unit and the controller; and a case having a door to open or close the chamber of the centrifugal device, further comprising a pump device having an air pump mounted in the case for vacuum and compression, and valves connected to the air pump and adapted to be automatically opened or closed to selectively perform vacuum or compression, the pump device being controlled by the controller, and a connector formed at a side of the case to connect the pump device with an external unit for performing vacuum and compression.

### Advantageous Effects

The centrifuge according to the present invention, comprises an air pump, valves, and controller to allow an external unit, connected to a case of the centrifuge via a connector, to perform vacuum and compression in addition to basic centrifugation, thereby sequentially performing liposuction, centrifugation, and lipoinjection processes for autologous fat grafting using a single instrument. The centrifuge of the present invention has the effect of eliminating a necessity of the installation space of a separate suction device, and of reducing the overall number of parts, resulting in a minimized centrifuge size. Further, by performing all the processes of autologous fat grafting using a single instrument, it is possible to enable rapid and convenient implementation of the processes, resulting in a reduced operation time and effort.

According to the present invention, furthermore, lipoinjection can be easily achieved using a pneumatic pressure even in the case of muscularis that are reluctant to the insertion of a syringe, and can ensure a constant injection pressure differently from conventional manual injection, thereby eliminating the risk of unevenness in the resultant fat suctioned or injected body regions. Through the use of speed adjustors, further, the operation speed of the centrifuge according to the present invention can be freely adjusted in consideration of the operator's skill.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is an elevation view illustrating the configuration of a centrifuge according to the present invention;

FIG. 2 is a flow chart illustrating the operation ready procedure of the centrifuge, immediately after power is initially supplied, according to the present invention;

FIG. 3 is a diagram illustrating the operational relationship of an air pump and valves for vacuum and compression included in the centrifuge according to the present invention; and

FIG. 4 is a flow chart illustrating the operating sequence of the centrifuge as controlled by foot switches according to the present invention.

### BEST MODE FOR CARRYING OUT THE INVENTION

Now, a preferred exemplary embodiment of the present invention will be explained in detail with reference to the accompanying drawings.

### Mode for the Invention

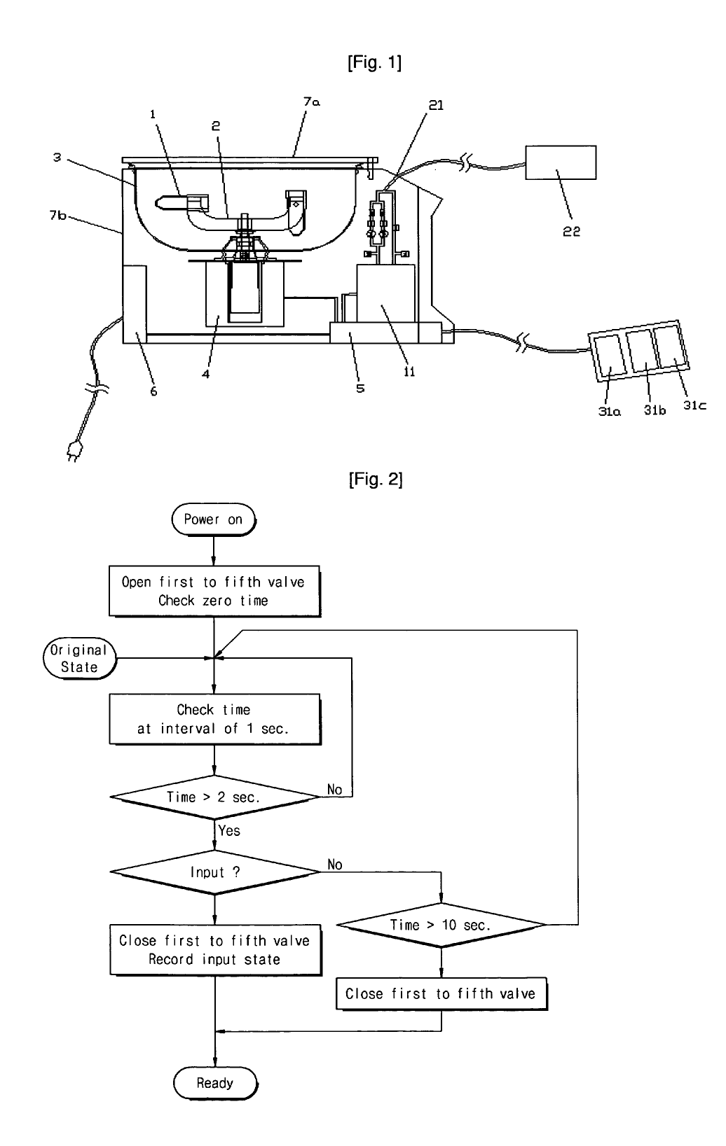

FIG. 1 is an elevation view illustrating a centrifuge according to the present invention. As shown in FIG. 1, the centrifuge comprises a centrifugal device 1, 2 and 3, a drive unit 4, a controller 5, a power supply 6, a case 7b, a pump device 11 and 12, and foot switches 31.

The centrifugal device includes a pair of buckets 1, a rotor 2 and a chamber 3, in the same manner as conventional centrifugal devices. The rotor 2 is a rotary wing, such as a swing rotor or angle rotor. The buckets 1 are mounted to the surface of the rotor 2, and one or more tubes or syringes are fitted into the buckets 1. The chamber 3 has an interior volume at least as wide as to stably or safely receive the rotating buckets and rotor 1 and 2.

The drive unit 4 is mounted to the lower end of the rotor 2 and is adapted to rotate the rotor 2 upon receiving electric power from the power source 6. The drive unit 4 is controlled by the controller 5.

A door 7a is provided at the top of the chamber 3 to close the chamber 3 for the safety of an operator when the rotor 2 rotates in the chamber 3. During a centrifugation process, the door 7a is opened to mount tubes or syringes, used to receive suctioned fat, to the buckets 1. Of course, the door 7a is closed prior to performing the centrifugation process.

The door 7a is pivotally hinged at a side of the case 7b as a supporting structure of the centrifuge. The case 7b is further provided with a connector 21 at a side thereof for the connection of an external unit 22.

The external unit 22, for example, is a fat injection vessel included in the sealed fat injection system disclosed in Korean Patent Application No. 10-2003-5029. Such an external unit 22 performs both liposuction and lipoinjection processes, thereby having no risk of external infection due to contact with the air.

The pump device includes an air pump 11 and a plurality of valves 12. The air pump 11 for vacuum and compression is mounted inside the case 7b and is connected to the connector 21. The valves 12 are interposed between the air pump 11 and the connector 21, and are designed to be automatically opened or closed to allow the external unit 22, connected with the connector 21, to selectively perform vacuum or compression. The pump device is also controlled by the controller 5.

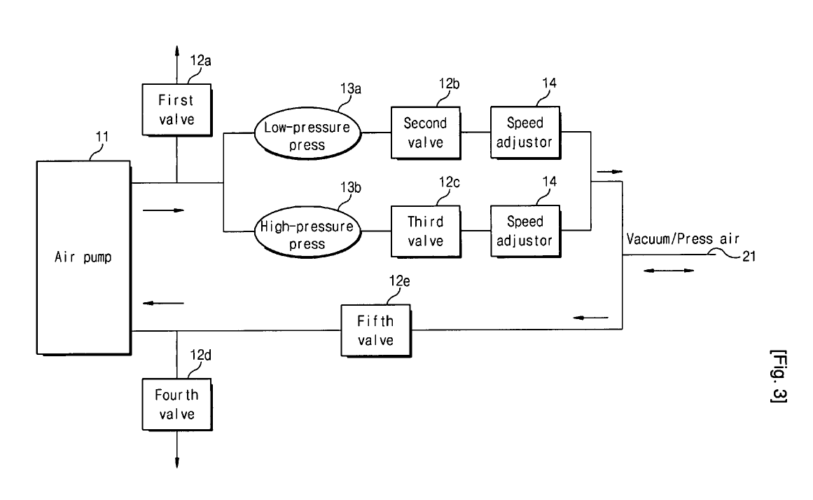

FIG. 2 is a flow chart illustrating the operation ready procedure of the centrifuge, immediately after power is initially supplied, according to the present invention. When the centrifuge is turned on upon receiving power from the power supply 6, the valves 12 of the pump device are simultaneously opened for 10 sec. by the controller 5. This procedure serves to discharge air remaining in lines between the connector 21 and the air pump 11 to prevent unwanted liposuction and lipoinjection by the external unit 22 due to a pneumatic pressure, thereby ensuring a safe surgical operation.

The operating time of the centrifuge is checked at an interval of 1 sec., and the centrifuge is programmed to have a rest time of 2 sec, which is the minimum air removal time for the driving of the air pump 11. If an operation start commend is inputted to the controller 5 after the lapse of 2 sec., the controller 5 simultaneously closes all the valves 12 and records an input state. After that, the controller 5 proceeds to a “ready” state as shown in FIG. 4 to operate the air pump 11 and the valves 12 for liposuction and lipoinjection. If no operating command is inputted, the controller 5 keeps the valves 12 in the opened state for 10 sec., and then automatically closes the valves 12. Then, the controller 5 proceeds to the ready state. Although the valve operating time of 10 sec. is sufficient for the removal of air, it may be appropriately changed without being limited thereto.

Although the centrifuge of the present invention configured as stated above is able to perform liposuction, centrifugation and lipoinjection processes, preferably, the centrifuge further comprises the foot switches 31 for facilitating the implementation of the liposuction and lipoinjection processes.

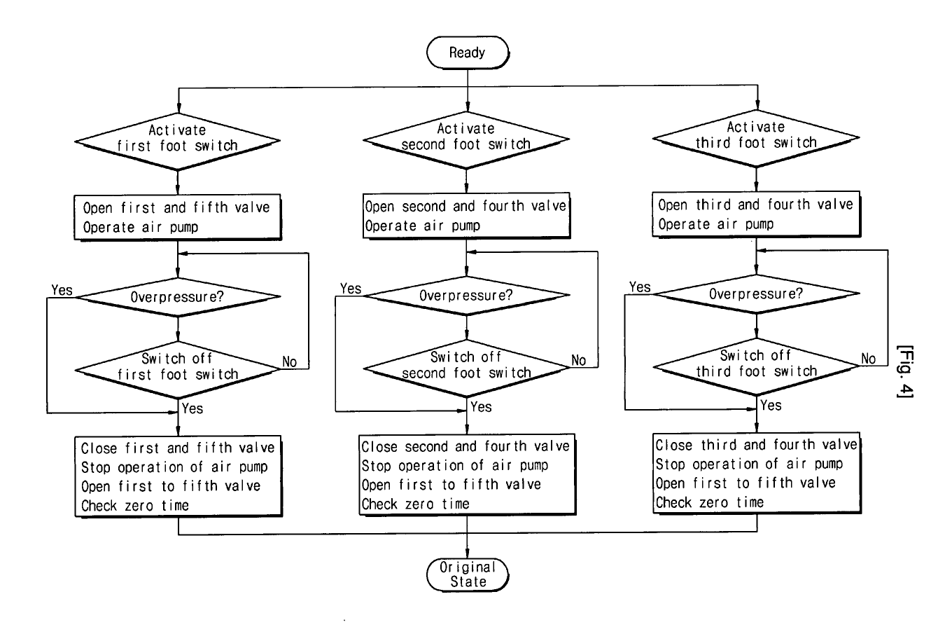

Now, the operating sequence of the centrifuge controlled by the foot switches and the operation of the air pump and valves for vacuum and compression will be explained with reference to FIGS. 3 and 4. In FIG. 3, arrows denote the flow of air.

The foot switches include a first foot switch 31a for vacuum, a second foot switch 31b for low-pressure compression, and a third foot switch 31c for high-pressure compression. The number of the foot switches may be varied in consideration of functions of the centrifuge.

In a liposuction process, first, a 2-5mm incision is made in the desired body regions for fat extraction, and a cannula is inserted through the incision. After that, the first foot switch 31a is activated to apply vacuum to the external unit 22 connected with the cannula. Thereby, first and fifth valves 12a and 12e are opened and the air pump 11 is operated by the controller 5. Through such an operating procedure, interior air of the external unit 22 is discharged to the outside by passing through the fifth valve 12e, air pump 11 and first valve 12a, resulting in a vacuum state in the external unit 22 for the liposuction process. To complete the liposuction process, the first foot switch 31a is switched off to close the first and fifth valves 12a and 12e and to stop the operation of the air pump 11.

It should be noted that the above described liposuction process accompanies the suction of blood, bodily fluids and other impurities in addition to suctioning the fat. If the mixture of the blood, bodily fluids, fat, and other impurities is directly injected into the desired body regions, such as the breasts, hips and face, the majority of the fat is absorbed into the remainder of the mixture, making it impossible to enlarge the desired body regions to a desired shape or size. For this reason, there have been many attempts to increase the survival rate of the fat, and as a result of clinical experimentation, it has been found that the survival rate of fat can be increased by injecting pure fat into the body regions. Such a separation of the pure fat is achieved by a centrifugation process that centrifugally separates the mixture of the blood, bodily fluids, impurities, fat, etc. into a blood layer and a fat layer by taking advantage of their different specific gravities. In this manner, after removal of the blood layer, pure fat, can be injected into the body.

After completing the centrifugation process, a cannula or syringe needle is inserted into the desired body regions for performing a lipoinjection process. For this, pressure is applied to the external unit 22 connected with the cannula or syringe needle.

The pressure is freely selectable within a wide range of pressures by means of pressure adjustors 13. The pressure adjustors 13 are pneumatically operable and are adapted to maintain a constant pressure. The use of the pressure adjustors enables the lipoinjection process to be performed at a constant pressure, thereby eliminating the risk of unevenness in the fat injected region. The pressure adjustors 13 include a low-pressure press 13a for use in the lipoinjection of subcutaneous fat layers, into which syringe needle is relatively easy, and a high-pressure press 13b for lipoinjection into muscularis that are reluctant to the insertion of the syringe needle.

Considering the lipoinjection process in relation with subcutaneous fat layers, first, the second foot switch 31b for low-pressure compression is activated to inject fat into subcutaneous fat layers. According to the activation of the second foot switch 31b, second and fourth valves 12b and 12d are opened and the air pump 11 is operated by the controller 5. Thereby, outside air is suctioned into the external unit 22 by passing through the fourth valve 12d, air pump 11, pressure adjustor 13, and second valve 12b. This allows the fat inside the external unit 22 to be injected into the desired body regions by way of the cannula or syringe needle. To complete the lipoinjection process, the second foot switch 31b is switched off to close the second and fourth valves 12b and 12d and to stop the operation of the air pump 11.

Next, considering the lipoinjection process in relation with muscularis that are reluctant to the insertion of the syringe needle, first, the third foot switch 31c for high-pressure compression is activated to ensure smooth injection of fat into the body region, namely, muscularis. According to the activation of, third and fourth valves 12c and 12d are opened and the air pump 11 is operated by the controller 5. Thereby, outside air is suctioned into the external unit 22 by passing through the fourth valve 12d, air pump 11, pressure adjustor 13, and third valve 12c. This allows the fat inside the external unit 22 to be injected into the desired body regions by way of the cannula or syringe needle. To complete the lipoinjection process, the third foot switch 31c is switched off to close the third and fourth valves 12c and 12d and to stop the operation of the air pump 11.

The centrifuge of the present invention further comprises speed adjustors 14 installed in press lines between the air pump 11 and the connector 21 to adjust the operational speed of the high-pressure or low-pressure compression. The speed adjustors 14 are pneumatically operable and can adjust the flow rate of air during the high-pressure or low-pressure compression in consideration of the operator's skill or desired body regions to be enlarged.

During the liposuction and lipoinjection processes, an interior pressure of the external unit 22 arrives an excessive pressure beyond a predetermined value determined by the pressure adjustor 13, the operation of the foot switches are stopped. That is, as shown in the flow chart of FIG. 4 illustrating the operating sequence of the centrifuge as controlled by the foot switches, if a response of a question overpressure? is yes, the air pump 11 and the valves 12 are automatically stopped in operation, causing the foot switches to be switched off. This is a safety measure against the malfunction of the pressure adjustor 13.

If the first to third foot switches 31 are switched off during the liposuction and lipoinjection processes, the valves 12 and the air pump 11 are stopped in operation, and the first to fifth valves 12 are opened simultaneously to remove the residual interior pressure of the external unit 22. This has the effect of preventing unwanted liposuction or lipoinjection due to a pneumatic pressure caused at a time that the cannula or syringe needle is removed from the body regions. In this way, the controller 5 returns to original state shown in FIG. 2, and repeats the above described procedure performed immediately after the centrifuge is turned on.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The centrifuge according to the present invention comprises an air pump, valves, and controller to allow an external unit, connected to a case of the centrifuge via a connector, to perform vacuum and compression in addition to basic centrifugation, thereby sequentially performing liposuction, centrifugation, and lipoinjection processes for autologous fat grafting using a single instrument. The centrifuge of the present invention has the effect of eliminating a necessity of the installation space of a separate suction device, and of reducing the overall number of parts, achieving a minimum centrifuge size. Further, by performing all the processes of autologous fat grafting using a single instrument, it is possible to enable rapid and convenient implementation of the processes, resulting in a reduced operation time and effort.

According to the present invention, furthermore, lipoinjection can be easily achieved using pneumatic pressure even in the case of muscularis that are reluctant to the insertion of a syringe, and can ensure a constant injection pressure differently from conventional manual injection, thereby eliminating the risk of unevenness in the resultant fat suctioned or injected body regions. Through the use of speed adjustors, further, the operation speed of the centrifuge according to the present invention can be freely adjusted in consideration of the operator's skill.

1. A centrifuge comprising: a centrifugal device having backets, a rotor, and a chamber; a drive unit to drive the rotor; a controller to control the drive unit; a power supply to supply power to the drive unit and the controller; and a case having a door open or close the chamber of the centrifugal device, further comprising:
a pump device having an air pump mounted in the case for vacuum and compression, and valves connected to the air pump and adapted to be automatically opened or closed to be selectively perform vacuum or compression, the pump device being controlled by the controller; and
a connector formed at a side of the case to connect the pump device with an external unit for performing vacuum and compression.

2. The apparatus as set forth in claim 1, further comprising:
pressure adjustors provided between the air pump and the connector of the case and adapted to adjust the level of a pressure for allowing liposuction and lipoinjection processes to be performed at a constant pressure.

3. The apparatus as set forth in claim 1, further comprising:
speed adjustors provided in press lines between the air pump and the connector of the case and adapted to adjust the flow rate of air in consideration of the skill of an operator and body regions where the liposuction and lipoinjection operations are performed.

4. A method of performing liposuction and lipoinjection using a centrifuge comprising: a centrifugal device having buckets, a rotor, and a chamber; a drive unit to drive the rotor; a case having a door to open or close the chamber of the centrifugal device; a pump device having an air pump mounted in the case for vacuum and compression, and valves connected to the air pump and adapted to be automatically opened or closed to selectively perform vacuum or compression, the valves including first to fifth valves; a controller to control the drive unit and the pump device; a power supply to supply power to the drive unit and the controller; and a connector formed at a side of the case to connect the pump device with an external unit for performing vacuum and compression, the method comprising:
performing a liposuction process by inserting a cannula into an incision and activating a first foot switch to open the first and fifth valves and to operate the air pump, applying vacuum to the external unit for liposuction;
performing a centrifugation process by mounting a syringe, that is filled with suctioned fat, and operating the centrifugal device to centrifugally obtain pure fat; and
performing a lipoinjection process by activating a second foot switch to open the second and fifth valves and to operate the air pump to thereby supply air into the external unit, allowing the pure fat inside the external unit to be injected into subcutaneous fat layers or by activating a third foot switch to open the third and fifth valves and to operate the air pump to thereby supply air into the external unit, allowing the pure fat inside the external unit to be injected into muscularis, wherein, prior to performing the processes, the method further comprises removing air remaining in lines between the connector of the case and the air pump by opening the valves by means of the controller at a time when the centrifuge is turned on upon receiving power from the power supply.

5. The method as set forth in claim 4, further comprising:
closing the valves and stopping the operation of the air pump if the controller detects an overpressure beyond a predetermined value during vacuum or compression.

6. The method as set forth in claim 4, further comprising:
removing a residual pressure inside the external unit by opening the valves after vacuum or compression is completed.

7. The method as set forth in claim 5, further comprising:
removing a residual pressure inside the external unit by opening the valves after vacuum or compression is completed.
